(19) United States
(12) Patent Application Publication
(10) Pub. No.: US 2010/0204050 A1
(43) Pub. Date: Aug. 12, 2010

(54) TARGET PREPARATION FOR PARALLEL SEQUENCING OF COMPLEX GENOMES

(76) Inventors: Horst Donner, Penzberg (DE); Bernd Buchberger, Zeitlarn (DE)

Correspondence Address: ROCHE DIAGNOSTICS OPERATIONS INC., 9115 Hague Road, Indianapolis, IN, 46250-0457, US

(21) Appl. No.: 12/689,269

(22) Filed: Jan. 19, 2010

Related U.S. Application Data
(63) Continuation of application No. PCT/EP2008/006030, filed on Jul. 23, 2008.

(30) Foreign Application Priority Data
Jul. 26, 2007 (EP) 07014641.0

(51) Int. Cl. C40B 20/02; C12Q 1/68; C40B 40/06

(52) U.S. Cl. 506/3; 435/6; 506/16

(57) ABSTRACT

The present invention provides a method for the isolation and analysis of a target nucleic acid, the target nucleic acid being present in a sample of genomic DNA, comprising the steps of a) fragmentation of the genomic DNA, b) hybridization of the genomic DNA on a nucleic acid solid support, the solid support comprising a plurality of oligonucleotide probes, the probes being characterized in that each probe is at least partially complementary to the sequence of the target nucleic acid or its complement, under hybridization conditions, characterized in that the plurality of probes hybridizes to fragments of the target nucleic acid but does not hybridize to other nucleic acids which are present in the sample, c) stripping off the target molecules hybridized to the nucleic acid array, d) overlap extension synthesis in order to generate double stranded overlap extension synthesis product, e) fragment polishing, and f) adaptor ligation.

### RELATED APPLICATIONS

This application is a continuation of PCT/EP2008/006030 filed Jul. 23, 2008 and claims priority to EP 07014641.0 filed Jul. 26, 2007.

### FIELD OF INVENTION

The present invention relates to the technical field of DNA sequence analysis. More specifically, the present invention relates to the technical field of enrichment of particular DNA sequences of interest that shall be subjected to a sequencing reaction subsequently.

### BACKGROUND OF THE INVENTION

There is currently no simple method allowing the sequencing of a substantial part of a complex genome (i.e. the human genome with 3 billion bases). However, individual eukaryotic genes (like EGF-receptor, size 110 kb) or gene cluster (like HLA, size 3 Mb) or the exon-parts of 100 to 400 disease-genes (oncogenes, variable size) are often in the range of 0.001% to 0.3% of the human genome, meaning 300 kb to 15 Mb. Isolation of the relevant DNA fragments from a genomic DNA preparation would take a high number of modified capture probes when using standard hybridization/capture approaches. Cloning of the relevant regions from individual patient samples is cumbersome and extremely time consuming. An amplification method for specific pre-amplification of regions in the Mb range is not available.

DNA sequencing has dramatically changed the nature of biomedical research and medicine. Reductions in the cost, complexity and time required to sequence large amount of DNA, including improvements in the ability to sequence bacterial and eukaryotic genomes will have significant scientific, economic and cultural impact.

New sequencing approaches like the GS20 instrument from 454 Life Science Corp. were developed for highly parallel sequencing with raw throughput significantly greater than that of state-of-the-art capillary electrophoresis instruments. The 454 apparatus uses a novel 60×60 mm2 fibreoptic slide containing 1,600,000 individual wells and is able to sequence 25 million bases, at 99% or better accuracy, in a 4 hour run. Recently this capacity has been increased by the GSFLX instrument to 100 million bases in one run. In order to avoid gaps in the DNA sequence to be analyzed, a 10 fold coverage for re-sequencing of complex genomes is recommended resulting in 10 Mb genomic DNA sequence obtained by a single run. Even this large amount of sequence information covers only 1/300 of the three billion bases of the complete human genome. It is therefore of interest to reduce the complexity of genomic nucleic acids from mammals to a size applicable for direct sequencing in this new sequencing instruments.

Reduction in complexity of nucleic acids can be achieved by different approaches. A PCR related approach is described by Telenius, H., et al., Genes Chromosomes Cancer 4 (1992) 257-263. In this approach, conserved regions in Alu repeat elements are used as primer binding sites in order to amplify regions between such elements. This approach leads to a loss of such elements which are conserved, highly repetitive and spread over the entire genome. Therefore the resulting PCR product is less complex compared with the original genomic DNA material. Yet, since the distribution of Alu repeats is not homogeneous, the amplification is biased and a complete coverage of the remaining genome is not achieved. Moreover, a selection of a specific region within the genome is not possible.

For enrichment of a specific region within a complex genome a hybridization of genomic DNA with primers selected from the region of interest and a subsequent capturing of this DNA utilizing a solid support was described in US 2006/0040300 and US 2006/0147940. This approach requires multiple oligonucleotides ranging in numbers from hundreds to thousands according to the size of the genomic region that has to be enriched. Synthesis of such large numbers of oligonucleotides is cost intensive and time consuming and parallel hybridization of large numbers of oligonucleotides to complex genomes is impossible to optimize for all oligonucleotides to the same extent. Therefore the complete coverage of the region of interest as well as the applicability to nucleic acid sequences of several Mb in size remains questionable.

A second aspect of the enrichment of specific portions of a complex genome is the loss of material during the process. A 300 fold depletion of the complexity of a full genome results in at least 300 fold less material left for further analysis. If for example, 1 μg of nucleic acid is required as template for parallel sequencing and the goal is a specific depletion of genomic DNA from 3000 Mb to 10 Mb, 300 μg of starting material is needed upfront. Therefore, an efficient reduction of the complexity requires additional amplification of the target nucleic acid in order to serve as template in highly parallel sequencing applications.

A related procedure is described in US 2006/0040300 and EP 1 645 640. Both procedures utilize a strand displacement polymerase (MDA—multiple displacement amplification) for subsequent amplification of the specifically enriched nucleic acid material. Strand displacement polymerases like the phi 29 polymerase use isothermal amplification at 37° C. and random priming for amplification of large nucleic acid stretches of more than 1 Mb (U.S. Pat. No. 5,001,050). A typical application is the amplification of whole genomes represented by DNA fragments of 30-50 kb in length, generated by standard DNA isolation procedures. The principle has its limitation related to the size and the type of nucleic acid (only genomic DNA>several kb) and the specificity of the amplification (random primers allows no specific amplification).

Another amplification method, PEP-PCR, is described by Zhang, L., et al., Proc. Natl. Acad. Sci 89 (1992) 5847-5851. This principle uses a 15 mer random primer and Taq polymerase for subsequent amplification of genomic DNA. Dean, F. B., et al., Proc. Natl. Acad. Sci. 99(8) (2002) 5261-5266, reports several drawbacks of this principle related to an amplification bias as well as the coverage of the complete sequence. Furthermore, a specific amplification is not feasible due to the use of random 15 mers.

In addition, for some specific situations it has also been disclosed that complexity reduction may also be achieved by means of hybridization onto nucleic acid arrays. WO 99/40194 discloses isolation of intermediate tandem repeat sequences from a sample by means of capturing them with a solid support. U.S. Pat. No. 6,828,104, discloses a method for depleting a sample from a plurality of sequences by means of incubating said sample onto a nucleic acid array, said array comprising a plurality of probes which are complementary to the plurality of sequences that shall become removed.

In conclusion, parallel sequencing expands the current limits in terms of capacity and therefore increases speed and reduces costs for sequencing applications. Nevertheless, the complete sequencing of complex genomes like mammalian genomes remains labor and cost intensive. Therefore a reduction of the complexity of such genomes to areas of interest is desirable.

Most technologies used so far are based on selective amplification via PCR or hybridization with specific probes and subsequent isolation via a solid support. The PCR based approaches are not free of amplification bias and not specific to certain areas or not suited for selective amplification of specific areas larger than several 100 kb. The hybridization technique requires many different probes and the depletion of large parts of the genomic sequence is also only useful in combination with an amplification principle. The best suited amplification principle is the whole genome amplification with strand-displacement polymerases like phi29. This amplification principle requires isothermal conditions and long nucleic acid fragments as template. The selective enrichment of specific nucleic acid sequences via hybridization and capturing onto a solid support is most efficient and specific when short nucleic acid fragments are used. In addition it needs to be considered that the more the genomic DNA is fragmented for efficient and specific hybridization, the more specific capture probes are needed for this approach. Therefore, a convenient workflow for simple target enrichment and preparation taking into account the specific target preparation requirements for the parallel sequencing approach is highly desirable. Thus, it was the goal of the present invention to elaborate a sample preparation method for selective sequencing in the range of one to several megabases out of (human) genomic DNA preparations

### SUMMARY OF THE INVENTION

In general, the present invention provides a method for the isolation and analysis of a target nucleic acid, said target nucleic acid being present in a sample of genomic DNA, comprising the steps of
a) fragmentation of said genomic DNA
b) specific hybridization of said genomic DNA on a nucleic acid solid support, said solid support comprising a plurality of oligonucleotide probes, said probes being characterized in that each probe is at least partially complementary to the sequence of said target nucleic acid or its complement under hybridization conditions characterized in that said plurality of probes hybridizes to fragments of the target nucleic acid but does not hybridize to other nucleic acids which are present in said sample,
c) stripping off the target molecules hybridized to said nucleic acid array
d) overlap extension synthesis in order to generate double stranded overlap extension synthesis product
e) fragment polishing
f) adaptor ligation

The term “hybridization” implies for a person skilled in the art that in order to obtain a specific hybridization result, a washing step is performed in order to remove all genomic DNA which does not specifically hybridize to said plurality of probes. Thus, step b) of the inventive method may also be defined as
b) hybridization of said genomic DNA on a nucleic acid solid support, said solid support comprising a plurality of oligonucleotide probes, said probes being characterized in that each probe is at least partially complementary to the sequence of said target nucleic acid or its complement under hybridization conditions characterized in that said plurality of probes hybridizes to fragments of the target nucleic acid but does not hybridize to other nucleic acids which are present in said sample, said hybridization comprising the step of removing all genomic DNA which is not specifically bound to said plurality of probes.

Preferably, said step of adaptor ligation is performed with exactly 2 adaptors A and B.

Also preferably, said nucleic acid solid support is a nucleic acid array.

Also preferably, step a) is performed by means of nebulization.

In one embodiment, the present invention further comprises the step of subsequent PCR amplification with amplification primers comprising sequences corresponding to said ligated adaptors.

Also according to the present invention, a single stranded DNA bead library may be generated subsequent to adaptor ligation, i.e. subsequent to step f) or subsequent to the PCR amplification.

It is also within the scope of the present invention, if said single stranded library is subjected to a sequencing reaction, preferably a sequencing by synthesis reaction and most preferably to a Pyrophosphate sequencing reaction.

The present invention is also directed to a kit comprising a nucleic acid solid support and at least one or more compounds from a group consisting of DNA Polymerase, T4 Polynucleotide Kinase, T4 DNA Ligase, a first blunt ended double stranded adaptor oligonucleotide, a second blunt ended double stranded adaptor oligonucleotide, an array hybridization solution, an array wash solution, and an array strip off solution. Preferably, said nucleic acid solid support is a nucleic acid array.

### BRIEF DESCRIPTION OF THE FIGURES

FIG. 1: Template preparation workflow for parallel sequencing applications on a GS FLX instrument, 454 Life Science Corporation USA. (Source: Page 12 GS DNA Library Preparation Kit User's Manual Version December 2006)

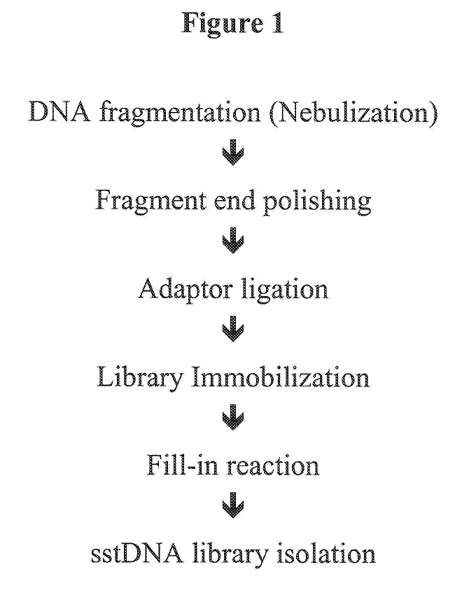

FIG. 2: Template preparation workflow according to example 1 for parallel sequencing applications on a GS FLX instrument, 454 Life Science Corporation USA, including a hybridization based sequence selection as well as subsequent ds DNA reconfiguration based on a overlap extension synthesis principle.

FIG. 3: Template preparation workflow according to example 2 for parallel sequencing applications on a GS FLX instrument, 454 Life Science Corporation USA, including a hybridization based sequence selection as well as subsequent ds DNA reconfiguration based on a overlap extension synthesis principle. Additionally with PCR amplification via linker sequence used for priming the sequencing reaction.

### DETAILED DESCRIPTION OF THE INVENTION

The present invention relates to template preparation method for sequencing applications of selected nucleic acid molecules with up to 10 Mb in length. The method consists of a capturing method for large sequence stretches of complex (i.e. mammalian) genomes and specific enzymatic target preparation steps required for generation target molecules for parallel sequencing applications. It combines an efficient capturing of fragmented genomic DNA by hybridization with oligonucleotides synthesized and ordered in an array fashion on a solid support with a convenient enzymatic sample preparation for parallel sequencing including subsequent template amplification.

The present invention relates to a method for target enrichment and preparation of specific nucleic acid molecules from complex genomes for subsequent analysis by parallel sequencing approaches. The detailed procedures in the examples are related to the 454 Life Science Corporation GS20 and GSFLX instrument (see GS20 Library Prep Manual, December 06, WO 2004/070007) but the principle is also applicable for other parallel sequencing approaches. It is useful for the selection of areas of interest from complex genomic DNA but also for the enrichment of other nucleic acid molecules derived from methylated DNA, mRNA or other parts of the transcriptome like miRNA.

The invention is directed to a procedure how to selectively purify relevant gene regions from a genomic DNA preparation and how to process such DNA to make it practicable as sample for high parallel sequencing. According to the invention, the genomic DNA is fragmented by mechanical stress. The desired average size of the DNA fragments is small (<5000 bp, preferably <=1000 bp) and depends on the sequencing method to be applied. For sequencing on a 454 GS20 or 454 FLX instrument, fragments should be in the range of 200 or 600 bp, respectively.

A first key aspect of the invention is to use high density DNA-arrays for specific, capturing of the gene regions of interest. Especially useful for this approach are NA-arrays produced by on-chip-synthesis. Such arrays can bear more than 1 million different capture sequences (features), can be produced flexible and target-specific at low price and in low numbers. Thus, in the context of the present invention, the term “plurality of oligonucleotide probes” is understood as comprising more than 100 and preferably more than 1000 oligonucleotides.

According to the manufacturing technology applied, the probe length of oligonucleotide hybridization probes, generated by on-chip synthesis can vary between 20 by to 70 by in length. The probe sequences are target specific and can be designed in silico utilizing appropriate probe sequence calculation algorithms.

The capturing comprises at first denaturation step, characterized in that the doubles stranded DNA is becoming single stranded upon thermal denaturation at around 85° C.-100° C. Then there follows the hybridization step, wherein the target DNA of interest is hybridized to a plurality of oligonucleotide hybridization probes. Subsequently, the single stranded DNA molecules bound to said oligonucleotide hybridization probes are stripped off by methods well known in the art into a solution that is subjected to further treatment steps.

Hybridization solutions generally contain 2.5 to 5×SSC or SSPE buffet. It also contains 0.1% to 0.25% sodium dodecylsulfate. In addition hybridization solutions may contain combinations of the following constituents: Up to 50% formamide, tRNA up to 1 mg/ml, Cot 1 DNA up to 2 mg/ml, BSA up to 0.3 mg per ml, Denhardt's solution, or up to 200 mg/ml salmon sperm DNA. Hybridization on the arrays is performed between 37° C. and up to 65° C. depending on the length of the oligonucleotide hybridization probes as well as the hybridization buffer composition.

A washing step is always performed in order to remove DNA which is not specifically hybridized. For array washing, a subsequent washing with different buffers of decreasing salt concentrations is performed. Typical washing buffers are 0.5×SSC and 1% SDS, 0.5×SSC, 0.1×SSC or 0.0.1×SSC buffers. The temperature during the washing steps can vary between 37° C. and 60° C. according to the length of the oligonucleotide hybridization probe as well as the washing buffer composition. Array stripping could be done for example under alkaline conditions (0.05 N NaOH for 10 min at 25° C.). The solution is then removed from the arrays and collected for further processing.

A second key aspect of the present invention is that after capturing of the single stranded DNA fragments corresponding to the target DNA of interest or its respective complement, the partially complementary single stranded DNA molecules are rehybridized and an overlap extension reaction is performed in order to generate a double stranded DNA molecules.

In another aspect, the sample processing workflow of the standard parallel sequencing approach has to be changed. In contrast to the standard 454 FLX instrument protocols, nucleic acids derived from array hybridization are single-stranded and have to be re-annealed to ds DNA fragments for further use in the parallel sequencing workflow.

As further key aspect, depending on the amount of starting material available or the final DNA amount actually needed, a sample amplification step is required in some instances, e.g. for sequencing from a small number of cells or even a single cell. It has to be considered, that the method according to the invention ends up with an amount of specific DNA, which is in the range of or well below 1/1000 of the starting amount.

The present invention includes the enrichment of specific nucleic acid molecules by hybridization with short oligonucleotides ranging from 15-80 by followed by capturing onto a solid support. Preferably, the oligonucleotides are coupled to said solid support by means of direct synthesis of these oligonucleotides on the solid support. Those solid supports are preferably flat and oligonucleotides are synthesized in an ordered array like fashion. Such oligonucleotide arrays are commercially available from Affymetrix Corporation, NimbleGen Corporation, Agilent Corporation and Combimatrix Corporation. Alternatively bead based arrays like those commercially available from Illumina Corporation can also be used.

In the context of the present invention, the following definitions shall apply:

Nucleic acid solid support: A nucleic acid solid support is a matrix, to which nucleic acids are coupled. The coupling is preferably a covalent bonding, but other non covalent bondings are also possible. The nucleic acids are double stranded or single stranded. Single stranded nucleic acids are highly preferred because they can be directly used as hybridization probes for binding any desired kind of hybridization partner. Most preferably, the single stranded nucleic acids or Poly-Desoxynucleotides with 5 and 100 nucleotide residues. A nucleic acid solid support with a flat matrix and a multitude of sites with each site comprising a different type of nucleic acid molecules is defined as a Nucleic Acid array.

DNA fragmentation: DNA fragmentation is defined as any method of generating small DNA fragments from a sample of genomic DNA. In the context of the present invention, fragments of about 50 by up to 1000 by are preferable. Highly preferred are fragments between 100 and 600 bp. Fragmentation can be achieved either enzymatically using frequently cutting restriction enzymes or physically, for example by means of shearing said DNA through a syringe, or by means of sonication. In particular, fragmentation may by achieved by means of nebulization (EP 0 552 290).

Isolation of a target nucleic acid: In the context of the present invention, “isolation of a target nucleic acid” shall be understood as a method of generating from a first sample of DNA, e.g. genomic DNA a second sample of DNA, in which at least one specific type of nucleic acid sequence is represented with a higher frequency as said particular type of sequence was represented in said first sample. In addition, such a method may include further processing steps of DNA manipulation and analysis such as fragment polishing, adaptor ligation and e.g. sequence determination.

“Stripping off”: “Stripping off” shall mean a process for removing single stranded nucleic acids which are hybridized on a solid support such as a nucleic acid array into an appropriate strip off solution. Preferably, this is achieved by means of a) applying an appropriate strip off solution, and b) temperature increase up to a temperature between 85° C. and 95° C. in order to dissolve the hybridization complexes formed between the capture probes of the nucleic acid array and the captured target nucleic acid molecules.

Overlap extension synthesis: Overlap extension synthesis is understood as one crucial element of the method according to the present invention. In this context it is understood that according to the present invention, the pool of single stranded target molecules which has been obtained by stripping off the target molecules from the solid support comprises multiple DNA molecules representing sequences of either the sense strand or the antisense strand of the target nucleic acid of interest. If in the beginning, a physical fragmentation method has been applied initially, none of the fragments has a defined length or end. Thus, as a prerequisite for overlap extension synthesis, the pool is incubated under conditions that intermolecular annealing may occur and partially complementary sense and antisense molecules may form hybrids with protruding single strands. The hybrids are then appropriately treated with a DNA dependent DNA polymerase in the presence of desoxynucleoside triphosphates. In specific embodiments, Klenow DNA polymerase or T4-DNA Polymerase may be used. In other embodiments, the step of overlap extension synthesis may be performed as the first cycle of an amplification protocol using a Taq DNA Polymerase or any other kind of thermostable DNA Polymerase.

Fragment polishing and adaptor ligation: In order to ligate blunt ended double stranded oligonucleotides onto a double stranded target molecule, it is necessary to ensure that this target molecule itself is blunt endend. In order to achieve this, the double stranded target molecules are subjected to a fill-in reaction with a DNA Polymerase such as T4-DNA Polymerase or Klenow polymerase in the presence of Desoxynucleoside Triphposphates, which results in blunt ended target molecules. In addition, T4 Polynucleotide Kinase is added prior to the ligation in order to add phosphate groups to the 5′ terminus, which are a prerequisite for the subsequent ligation step. Subsequent ligation of the adaptors (short double stranded blunt end DNA oligonucleotides with about 3-20 base pairs) onto the polished target DNA may be performed according to any method which is known in the art, preferably by means of a T4-DNA ligase reaction.

In the context of the present invention, it is particularly advantageous, if 2 different types of double stranded adaptor molecules A ad B with different sequences are used. This allows for the generation of a single stranded library, characterized in that each single strand has a first end comprising a sequence according to the A adaptor and a second sequence corresponding to the B adaptor. If at least one adaptor in addition carries a modification entity such as Biotin, then the captured target molecules may be subsequently bound on a solid support, such as a Streptavidin coated bead.

Single stranded DNA library: A single stranded DNA library is defined as a plurality of different single stranded DNA molecules. A single stranded DNA bead library in the context of the present invention is understood as a single stranded library characterized in that each single stranded DNA molecule is non covalently attached to a bead. For example, if adaptors comprising a modification, e.g. a Biotin modification as disclosed above are being used, a respective single stranded library can be attached to Streptavidin coated beads.

Sequencing by synthesis: Sequencing by synthesis according to the literature in the art is defined as any sequencing method which monitors the generation of side products upon incorporation of a specific Desoxynucleoside-Triphopshate during the sequencing reaction. One particular and most prominent embodiment of the sequencing by synthesis reaction is the pyrophosphate sequencing method. In this case, generation of pyrophosphate during nucleotide incorporation is monitored by means of an enzymatic cascade which finally results in the generation of a chemo-luminescent signal. For example, the 454 Genome sequencer System (Roche Applied Science cat. No. 04 760 085 001) is based on the pyrophosphate sequencing technology.

Summarizing, a typical workflow of the present invention for the isolation and sequence analysis of a target nucleic acid, said target nucleic acid being present in a sample of genomic DNA, comprising the steps of
a) fragmentation of said genomic DNA, preferably by means of nebulization in order to obtain unbiased results
b) hybridization of said genomic DNA on a nucleic acid solid support, said solid support comprising a plurality of oligonucleotide probes, said probes being characterized in that each probe is al east partially complementary to the sequence of said target nucleic acid or its complement, under hybridization conditions, characterized in that said plurality of probes hybridizes to fragments of the target nucleic acid but does not hybridize to other nucleic acids which are present in said sample,
c) stripping off the target molecules hybridized to said nucleic acid array
d) overlap extension synthesis in order to generate double stranded overlap extension synthesis product
e) fragment polishing,
f) adaptor ligation, preferably with 2 adaptors A and B

Step b) needs to be performed in such a way that the genomic DNA is first denatured preferably by means of heating into single stranded molecules prior to hybridization onto the nucleic acid solid support. Then, stripping according to step c) results in a pool of single stranded target molecules. Subsequent to the stripping, re-hybridization of single strands occurs not only between strands of equal length and absolute complementarity, but also between strands that just have a partial overlap. Thus, overlap extension according to step d) is required.

Said nucleic acid solid support is preferably a nucleic acid array. The oligonucleotide probes of said array may have been synthesized in situ on said array by standard methods known in the art.

Optionally, a PCR amplification with amplification primers comprising sequences corresponding to said ligated adaptors may be performed subsequently to the adaptor ligation.

Also according to the present invention, a single stranded DNA bead library may be generated subsequent to adaptor ligation. generation of such a single stranded DNA bead library may be done according to the Genome Sequencer Workflow as basically disclosed in the manual of the Genome Sequencer FLX instrument (Roche Applied Science Catalog No. 04 896 548 001). If two adaptors A and B are used, 3 types target molecules can be discriminated subsequently:
(i) molecules with one A and one B adaptor
(ii) molecules with 2 A adaptors
(iii) molecules with 2 B adaptors

If one of said adaptors, e.g. adaptor A carries a biotin modification, then molecules (i) and (ii) can be bound on streptavidin coated magnetic particles for further isolation. Subsequently, the double stranded DNA molecules that have been bound to said magnetic particles are thermally denatured in such a way that only molecules comprising one A and one B adaptor are released into solution. (Due to the tight Biotin/Streptavidin bonding, molecules with 2 A adaptors only will not be released into solution). Said solution comprising single stranded target molecules with an A adaptor at one end and a B adaptor at the other end can subsequently be bound on a further type of beads comprising a capture sequence which is sufficiently complementary to the adaptor B sequence for further processing.

Further according to the present invention, said single stranded library may be subjected to a sequencing reaction, preferably a sequencing by synthesis reaction and most preferably to a pyrophosphate sequencing reaction. In case of the Genome Sequencer workflow (Roche Applied Science Catalog No. 04 896 548 001), in a first step, clonal amplification is performed by means of emulsion PCR. The beads carrying the clonally amplified target nucleic acids are then arbitrarily transferred into a picotiter plate and subjected to a pyrophosphate sequencing reaction for sequence determination.

The following examples, references and figures are provided to aid the understanding of the present invention, the true scope of which is set forth in the appended claims. It is understood that modifications can be made in the procedures set forth without departing from the spirit of the invention.

### EXAMPLES

Exemplary workflows are disclosed in more detail in the following examples that might relate to different sequencing applications. According to the examples an amplification of the target nucleic acid after hybridization, reassembly and adaptor ligation using PCR based techniques is described.

### Example 1

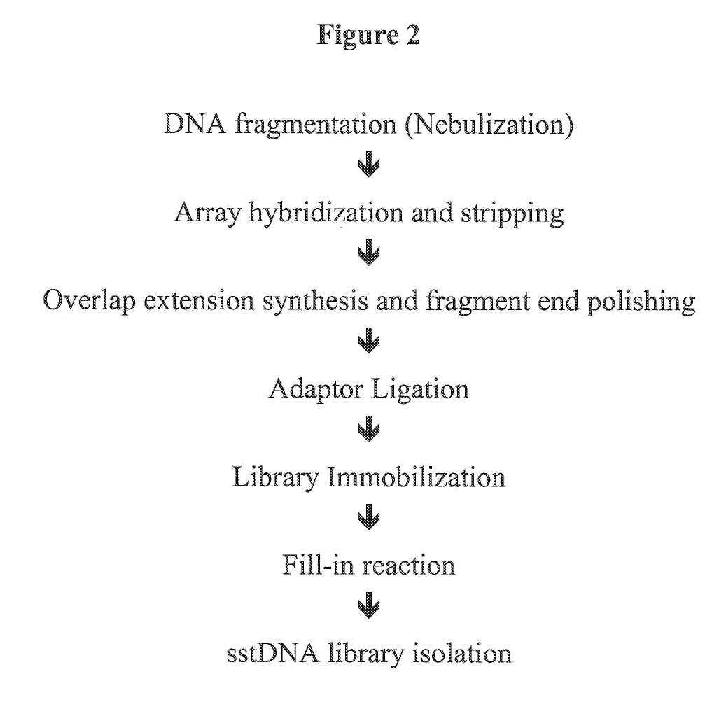

Example 1 is related to the enrichment of specific portions of genomic DNA according to the procedure described in FIG. 2. This example uses high amounts of input sample DNA and therefore do not include an amplification step during single stranded DNA library preparation. Specific DNA is captured as random single strand fragments on the chip. The chip has to carry capture probes for both strands. After washing and elution complementary strands are hybridized, the resulting overhangs are filled and the ends are polished. Now the protocol proceeds with the standard method starting with the step of linker ligation.

### Fragmentation:

Obtain 1-100 μg of sample DNA (in TE) and pipette it to the bottom (cup) of a Nebulizer.

Add TE Buffer to a final volume of 100 μl.

Add 500 μl of Nebulization Buffer and mix thoroughly by swirling or pipetting up and down.

Apply the sample to fragmentation.

Total recovery should be greater than 300 μl.

Add 2.5 ml of Qiagen's Buffer PB directly into the Nebulizer cup and swirl to collect all material droplets and mix the sample.

Purify the nebulized DNA using two columns from a MinElute PCR Purification Kit (Qiagen), according to the manufacturer's instructions for spin columns using a microcentrifuge, with the following exceptions:

The large sample volume (after the addition of Qiagen's Buffer PB) will require that each column be loaded and spun in two aliquots (approx. 750 μl each).

After the PE dry spin, rotate the column 180° and spin an additional 30 seconds to ensure complete removal of the ethanol.

Elute with 25 μl of Buffer EB (room temperature; supplied in the Qiagen kit).

Pool the eluates of the two columns, for a total volume of ˜50 μl.

### Small Fragment Removal:

Add Buffer EB (Qiagen) to a final volume of 50 μl.

Add 35 μl of AMPure SPRI beads. Vortex to mix.

Incubate for 5 minutes at room temperature (22° C.).

Using a Magnetic Particle Collector (MPC), pellet the beads against the wall of the tube (this may take several minutes due to the high viscosity of the solution.)

Leave the tube of beads in the MPC during all wash steps.

Remove the supernatant and wash the beads twice with 500 μl of 70% ethanol.

Small, sub-quality DNA fragments (<250 bp) do not bind to SPRI beads under the incubation conditions used, and will be washed away.

Remove all the supernatant and allow the SPRI beads to air dry completely. The drying time can vary due to environmental conditions and the amount of residual fluid left in the tube. The tube may be placed in a heating block set to 37° C. to help speed drying; the beads are dry when visible cracks form in the pellet.

Remove the tube from the MPC, add 24 μl of 10 mM Tris-HCl, pH 8.0 (or Qiagen's Buffer EB), and vortex to re-suspend the beads. This elutes the fragmented DNA from the SPRI beads.

Using the MPC, pellet the beads against the wall of the tube once more, and transfer the supernatant containing your purified fragmented DNA to a fresh microcentrifuge tube.

### Array Hybridization and Stripping:

Suspend the fragmented DNA in an array hybridization buffer. Depending on the array used the hybridization buffer could be salt buffer with or without formamide.

According to the array used, 1 to 100 μg of fragmented DNA is hybridized to the arrays. Depending on the hybridization conditions (i.e. agitation or not, temperature, etc.) 3 h up to 3 days of hybridization time are required.

After hybridization, the hybridization solution is removed from the arrays followed by subsequent washing of the arrays using buffers with decreasing salt concentrations.

Bound target molecules are removed from the arrays by stripping under denaturating conditions. A procedure for complete removal of targets from DNA microarrays of different vendors is published by Hahnke, K., et al., J. Biotechnol 128 (2007) 1-13.

Removed targets are collected and subjected to a cleanup procedure using

At this point, the SUPERNATANT contains the single-stranded template DNA.

Carefully remove and transfer the SUPERNATANT to the freshly-prepared neutralization solution.

Repeat steps for a total of two 50 μl Melt Solution washes of the beads (pooled together into the same tube of neutralization solution).

Purify the neutralized ssDNA using one column from a MinElute PCR

Purification Kit (Qiagen). Follow the manufacturer's instructions for spin columns using a microcentrifuge, with the following exceptions:
a. After the PE dry spin, rotate the column 180° and spin an additional 30 seconds to ensure complete removal of the ethanol.
b. Elute with 15 μl of TE Buffer (from the GS DNA Library Preparation Kit; room temperature).

### Overlap Extension Synthesis and Fragment End Polishing:

Re-annealing of complex DNA fragment mixtures results in ds DNA fragments with 3′ and 5′ overhanging ssDNA ends. To generate integer ds DNA molecules, add the following reagents, in the order indicated:
˜23 μl purified, DNA fragments
5 μl 10× Polishing Buffer
5 μl BSA
5 μl ATP
2 μl dNTPs*
5 μl T4 PNK
5 μl T4 DNA polymerase
50 μl final volume

Mix well and incubate the polishing reaction for 15 minutes at 12° C.

Immediately continue incubation at 25° C. for an additional 15 minutes.

Purify the polished fragments using one column from a MinElute PCR Purification Kit (Qiagen), according to the manufacturer's instructions for spin columns using a microcentrifuge, with the following exceptions:
a. After the PE dry spin, rotate the column 180° and spin an additional 30 seconds to ensure complete removal of the ethanol.
b. Elute with 15 μl of Buffer EB (room temperature).

### Adaptor Ligation:

In a microcentrifuge tube, add the following reagents, in the order indicated:
˜15 μl Polished DNA
20 μl 2× Ligase Buffer
1 μl Adaptors
4 μl Ligase
40 μl total

Mix well, spin briefly, and incubate the ligation reaction at 25° C. for 15 minutes.

Purify the ligation products using one column from a MinElute PCR Purification Kit (Qiagen), according to the manufacturer's instructions for spin columns using a microcentrifuge, with the following exceptions:
a. After the PE dry spin, rotate the column 180° and spin an additional 30 seconds to ensure complete removal of the ethanol.
b. Elute with 25 μl of Buffer EB (room temperature), directly into the washed Library Immobilization Beads.

### Library Immobilization:

Transfer 50 μl of Library Immobilization Beads to a fresh 1.5 ml tube.

Using a Magnetic Particle Collector (MPC), pellet the beads and remove the buffer.

Wash the Library Immobilization Beads twice with 100 μl of 2× Library Binding Buffer, using the MPC.

Resuspend the beads in 25 μl of 2× Library Binding Buffer.

Elute the ligated DNA from the MinElute column (25 μl) directly into the tube of washed Library Immobilization Beads.

Mix well and place on a tube rotator at room temperature (+15 to +25° C.) for 20 minutes.

Using the MPC, wash the immobilized Library twice with 100 μl of Library Wash Buffer.

### Fill-In Reaction:

In a 1.5 ml tube, add the following reagents, in the order indicated, and mix:
40 μl Molecular Biology Grade water
5 μl 10× Fill-in Polymerase Buffer
2 μl dNTP Mix
3 μl T4 Polymerase
50 μl total

Using the MPC, remove the 100 μl of Library Wash Buffer from the Library-carrying beads.

Add the 50 μl of fill-in reaction mix.

Mix well and incubate at 37° C. for 20 minutes.

Using the MPC, wash the immobilized Library twice with 100 μl of Library Wash Buffer.

Single-Stranded Template DNA (sstDNA) Library Isolation:

In a 1.5 ml tube, prepare the neutralization solution by mixing 500 μl of Qiagen's PB buffer and 3.8 μl of 20% acetic acid.

Using the MPC, remove the 100 μl of Library Wash Buffer from the Library-carrying beads.

Add 50 μl of Melt Solution to the washed Library-carrying beads.

Vortex well and using the MPC, pellet the beads away from the 50 μl supernatant.

At this point, the SUPERNATANT contains the single-stranded template DNA (sstDNA) library.

Carefully remove and transfer the SUPERNATANT to the freshly-prepared neutralization solution.

Repeat steps for a total of two 50 μl Melt Solution washes of the beads (pooled together into the same tube of neutralization solution).

Purify the neutralized sstDNA library using one column from a MinElute PCR Purification Kit (Qiagen). Follow the manufacturer's instructions for spin columns using a microcentrifuge, with the following exceptions:
a. After the PE dry spin, rotate the column 180° and spin an additional 30 seconds to ensure complete removal of the ethanol.
b. Elute with 15 μl of TE Buffer (from the GS DNA Library Preparation Kit; room temperature).

### Example 2

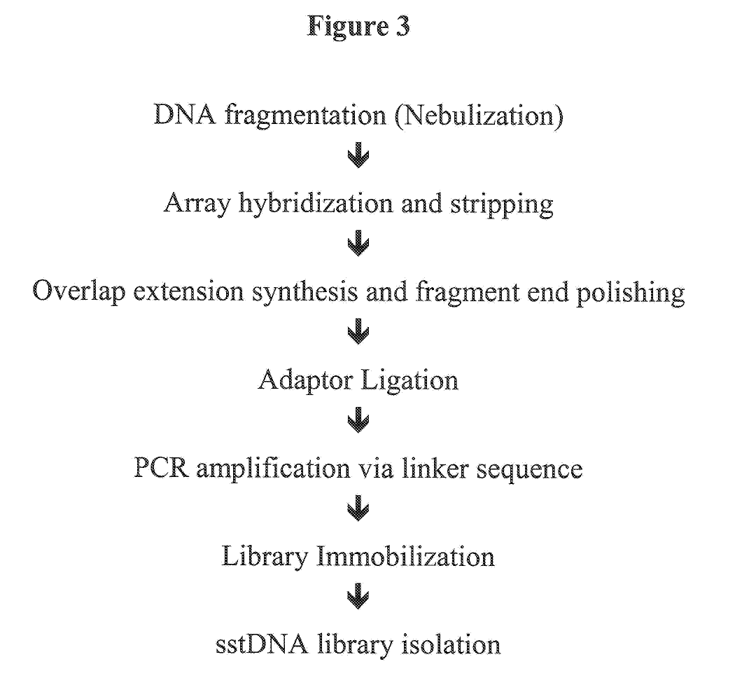

Example 2 is related to the enrichment of specific portions of genomic DNA including PCR based amplification according to the procedure described in FIG. 3. This variant requires less starting material since a PCR based amplification is included in the workflow. Until the step of linker ligation this protocol is identical to variant 1. After adapter ligation the double-stranded target DNA is amplified via PCR (5-25 cycles) using the adapter sequences as priming sites.

### Fragmentation:

Obtain 0.5-5 μg of sample DNA (in TE) and pipette it to the bottom (cup) of a Nebulizer.

Add TE Buffer to a final volume of 100 μl.

Add 500 μl of Nebulization Buffer and mix thoroughly by swirling or pipetting up and down.

Apply the sample to fragmentation.

Total recovery should be greater than 300 μl.

Add 2.5 ml of Qiagen's Buffer PB directly into the Nebulizer cup and swirl to collect all material droplets and mix the sample.

Purify the nebulized DNA using two columns from a MinElute PCR Purification Kit (Qiagen), according to the manufacturer's instructions for spin columns using a microcentrifuge, with the following exceptions:

The large sample volume (after the addition of Qiagen's Buffer PB) will require that each column be loaded and spun in two aliquots (approx. 750 μl each).

After the PE dry spin, rotate the column 180° and spin an additional 30 seconds to ensure complete removal of the ethanol.

Elute with 25 μl of Buffer EB (room temperature; supplied in the Qiagen kit).

Pool the eluates of the two columns, for a total volume of ˜50 μl.

### Small Fragment Removal:

Add Buffer EB (Qiagen) to a final volume of 50 μl.

Add 35 μl of AMPure SPRI beads. Vortex to mix.

Incubate for 5 minutes at room temperature (22° C.).

Using a Magnetic Particle Collector (MPC), pellet the beads against the wall of the tube (this may take several minutes due to the high viscosity of the solution.)

Leave the tube of beads in the MPC during all wash steps.

Remove the supernatant and wash the beads twice with 500 μl of 70% Ethanol.

Small, sub-quality DNA fragments (<250 bp) do not bind to SPRI beads under the incubation conditions used, and will be washed away.

Remove all the supernatant and allow the SPRI beads to air dry completely. The drying time can vary due to environmental conditions and the amount of residual fluid left in, the tube. The tube may be placed in a heating block set to 37° C. to help speed drying; the beads are dry when visible cracks form in the pellet.

Remove the tube from the MPC, add 24 μl of 10 mM Tris-HCl, pH 8.0 (or Qiagen's Buffer EB), and vortex to re-suspend the beads. This elutes the fragmented DNA from the SPRI beads.

Using the MPC, pellet the beads against the wall of the tube once more, and transfer the supernatant containing your purified fragmented DNA to a fresh microcentrifuge tube.

### Array Hybridization and Stripping:

Suspend the fragmented DNA in an array hybridization buffer. Depending on the array used the hybridization buffer could be salt buffer with or without formamide.

According to the array used 1 to 100 μg of fragmented DNA is hybridized to the arrays. Depending on the hybridization conditions (i.e. agitation or not, temperature, etc.) 3 h up to 3 days of hybridization time are required.

After hybridization, hybridization solution is removed from the arrays followed by subsequent washing of the arrays using buffers with decreasing salt concentrations.

Bond target molecules are removed from the arrays by stripping under denaturating conditions. A procedure for complete removal of targets from DNA microarrays of different vendors is published by Hahnke, K., et al., J. Biotechnol 128 (2007) 1-13.

Removed targets are collected and subjected to a cleanup procedure.

At this point, the SUPERNATANT contains the single-stranded template DNA.

Carefully remove and transfer the SUPERNATANT to the freshly-prepared neutralization solution.

Repeat steps for a total of two 50 μl Melt Solution washes of the beads (pooled together into the same tube of neutralization solution).

Purify the neutralized ssDNA using one column from a MinElute PCR.

Purification Kit (Qiagen). Follow the manufacturer's instructions for spin columns using a microcentrifuge, with the following exceptions:
a. After the PE dry spin, rotate the column 180° and spin an additional 30 seconds to ensure complete removal of the ethanol.
b. Elute with 15 μl of TE Buffer (from the GS DNA Library Preparation Kit; room temperature).

### Overlap Extension Synthesis and Fragment End Polishing:

Re-annealing of complex DNA fragment mixtures results in ds DNA fragments with 3′ and 5′ overhanging ssDNA ends. To generate integer ds DNA molecules, add the following reagents, in the order indicated:
˜23 μl purified, DNA fragments
5 μl 10× Polishing Buffer
5 μl BSA
5 μl ATP
2 μl dNTPs*
5 μl T4 PNK
5 μl T4 DNA polymerase
50 μl final volume

Mix well and incubate the polishing reaction for 15 minutes at 12° C.

Immediately continue incubation at 25° C. for an additional 15 minutes.

Purify the polished fragments using one column from a MinElute PCR Purification Kit (Qiagen), according to the manufacturer's instructions for spin columns using a microcentrifuge, with the following exceptions:
a. After the PE dry spin, rotate the column 180° and spin an additional 30 seconds to ensure complete removal of the ethanol.
b. Elute with 15 μl of Buffer EB (room temperature).

### Adaptor Ligation:

In a microcentrifuge tube, add the following reagents, in the order indicated:
˜15 μl Polished DNA
20 μl 2× Ligase Buffer
1 μl Adaptors
4 μl Ligase
40 μl total

Mix well, spin briefly, and incubate the ligation reaction at 25° C. for 15 minutes.

Purify the ligation products using one column from a MinElute PCR Purification Kit (Qiagen), according to the manufacturer's instructions for spin columns using a microcentrifuge, with the following exceptions:

a. After the PE dry spin, rotate the column 180° and spin an additional 30 seconds to ensure complete removal of the ethanol.

### Pcr Amplification Via Linker Sequences:

Linker sequences are used as primer binding sites for subsequent amplification of the target molecules.

Prepare the following mixes:
1 μl PCR Grade Nucleotide Mix
5 μl PCR primer mix, 10×
25 μl Template DNA, 0.1 ng-250 ng
4 μl Water, PCR Grade
   and
9.25 μl sterile double-dist. water
5 μl Expand High Fidelity buffer 10×
0.75 μl Expand High Fidelity Enzyme mix Taq/Tgo)
Final volume 50 μl.

Combine Mix 1 and Mix 2 in a thin-walled PCR tube (on ice). Gently vortex the mixture to produce a homogeneous reaction, then centrifuge briefly to collect sample at the bottom of the tube.

Run the following program on a thermocycler:

| | | | |
|---|---|---|---|
| Initial Denaturation | 94° | C. | 2 min. |
| 1 cycle | | | |
| Denaturation | 94° | C. | 15 sec. |
| Annealing | 45-65° | C. | 30 sec (temperature depending |
| | | | on primer used) |
| Elongation | 72° | C. | 45 sec. |
| 10 cycles | | | |
| Denaturation | 94° | C. | 15 sec. |
| Annealing | 45-65° | C. | 30 sec (temperature depending |
| | | | on primer used) |
| Elongation | 72° | C. | 45 sec + 5 sec per cycle |
| 10-15 cycles | | | |
| Final Elongation | 72° | C. | 7 min. |
| Cooling | 4° | C. | |

Purify the PCR products using one column from a MinElute PCR Purification Kit (Qiagen), according to the manufacturer's instructions for spin columns using a microcentrifuge, with the following exceptions:
a. After the PE dry spin, rotate the column 180° and spin an additional 30 seconds to ensure complete removal of the ethanol.
b. Elute with 15 μl of Buffer EB (room temperature), directly into the washed Library Immobilization Beads.

### Library Immobilization:

Transfer 50 μl of Library Immobilization Beads to a fresh 1.5 ml tube.

Using a Magnetic Particle Collector (MPC), pellet the beads and remove the buffer.

Wash the Library Immobilization Beads twice with 100 μl of 2× Library Binding Buffer, using the MPC.

Resuspend the beads in 25 μl of 2× Library Binding Buffer.

Elute the ligated DNA from the MinElute column (25 μl; directly into the tube of washed Library Immobilization Beads.

Mix well and place on a tube rotator at room temperature (+15 to +25° C.) for 20 minutes.

Using the MPC, wash the immobilized Library twice with 100 μl of Library Wash Buffer.

Single-Stranded Template DNA (sstDNA) Library Isolation:

In a 1.5 ml tube, prepare the neutralization solution by mixing 500 μl of Qiagen's PB buffer and 3.8 μl of 20% acetic acid.

Using the MPC, remove the 100 μl of Library Wash Buffer from the Library-carrying beads.

Add 50 μl of Melt Solution to the washed Library-carrying beads.

Vortex well and using the MPC, pellet the beads away from the 50 μl supernatant.

At this point, the SUPERNATANT contains the single-stranded template DNA (sstDNA) library.

Carefully remove and transfer the SUPERNATANT to the freshly-prepared neutralization solution.

Repeat steps for a total of two 50 μl Melt Solution washes of the beads (pooled together into the same tube of neutralization solution).

Purify the neutralized sstDNA library using one column from a MinElute PCR Purification Kit (Qiagen). Follow the manufacturer's instructions for spin columns using a microcentrifuge, with the following exceptions:
a. After the PE dry spin, rotate the column 180° and spin an additional 30 seconds to ensure complete removal of the ethanol.
b. Elute with 15 μl of TE Buffer (from the GS DNA Library Preparation Kit; room temperature).

What is claimed is:

1. A method for isolation and analysis of a target nucleic acid, the target nucleic acid being present in a sample of genomic DNA, comprising the steps of
(a) fragmentation of the genomic DNA,
(b) hybridization of the genomic DNA on a nucleic acid solid support, the solid support comprising a plurality of oligonucleotide probes, wherein each probe is at least partially complementary to the target nucleic acid or its complement under hybridization conditions wherein the plurality of probes hybridizes to fragments of the target nucleic acid but does not hybridize to other nucleic acids present in the sample,
(c) stripping off the target molecules hybridized to the nucleic acid support,
(d) overlap extension synthesis in order to generate a double stranded overlap extension synthesis product,
(e) fragment polishing, and
(f) adaptor ligation of exactly two adaptors A and B.

2. The method according to claim 1, wherein the nucleic acid solid support is a nucleic acid array.

3. The method according to claim 1, wherein step (a) is performed by means of nebulization.

4. The method according to claim 1, further comprising the step of
(g) PCR amplification with amplification primers comprising sequences corresponding to the ligated adaptors.

5. The method according to claim 1, subsequent to step (f), further comprising the step of generating a single stranded DNA bead library.

6. The method according to claim 5, wherein the single stranded library is subjected to a sequencing reaction, preferably a sequencing by synthesis reaction, and most preferably to a pyrophosphate sequencing reaction.

7. The method according to claim 4, subsequent to step (g), further comprising the step of generating a single stranded DNA bead library.

8. The method according to claim 7, wherein the single stranded library is subjected to a sequencing reaction.

9. The method according to claim 7, wherein the single stranded library is subjected to a sequencing by synthesis reaction.

10. The method according to claim 7, wherein the single stranded library is subjected to a pyrophosphate sequencing reaction.

11. A kit for isolation and analysis of a target nucleic acid according to claim 1, the kit comprising a nucleic acid solid support and one or more selected from the group consisting of DNA polymerase, T4 polynucleotide kinase, T4 DNA ligase, a first blunt ended double stranded adaptor oligonucleotide, a second blunt ended double stranded adaptor oligonucleotide, an array hybridization solution, an array wash solution, and an array strip off solution.

12. The kit according to claim 11, wherein the nucleic acid solid support is a nucleic acid array.
